(19) United States
(12) Patent Application Publication
(10) Pub. No.: US 2017/0007392 A1
(43) Pub. Date: Jan. 12, 2017

(54) ENDOPROSTHESIS FOR ENDOVASCULAR TREATMENT OF THORACIC-ABDOMINAL AORTIC ANEURYSMS OR DISSECTIONS AND ENDOPROSTHESIS FOR ENDOVASCULAR TREATMENT OF ABDOMINAL AORTIC ANEURYSMS OR DISSECTIONS WHICH COMPROMISE THE ILIAC ARTERIES

(71) Applicant: BIOKYRA PESQUISA E DESENVOLVIMENTO LTDA., Florianópoilis, SC (BR)

(72) Inventors: Marco Antonio LOURENÇO, Curitiba, PR (BR); Charles Cristian Facchini de Souza, Florianópolis, SC (BR); Isaias Masiero Filho, Florianópolis, SC (BR)

(73) Assignee: BIOKYRA PESQUISA E DESENVOLVIMENTO LTDA., Florianópolis, SC (BR)

(21) Appl. No.: 15/113,302

(22) PCT Filed: Jan. 23, 2014
(86) PCT No.: PCT/BR2014/000043

§ 371 Date: Jul. 21, 2016

(51) Int. Cl. A61F 2/07; A61F 2/856

(52) U.S. Cl. CPC A61F 2/07; A61F 2/856; A61F 2002/061

(57) ABSTRACT

The present invention relates to an endoprosthesis for endovascular treatment of thoracic-abdominal aortic aneurysms or dissections, wherein the endoprosthesis has a proximal region, an intermediate region and a distal region, comprising five inner cylinders parallel to the longitudinal axis of the endoprosthesis, said inner cylinders, having elliptical apertures in the intermediate region. The present invention also relates to an endoprosthesis for endovascular treatment of abdominal aortic aneurysms or dissections which compromise the iliac arteries, wherein the endoprosthesis has a proximal region, an intermediate region and a distal region, comprising an inner cylinder parallel to the longitudinal axis of the endoprosthesis, said inner cylinder having an elliptical aperture in the intermediate region.

### FIELD OF APPLICATION

The present invention belongs to the field of prostheses implantable inside the human body, especially to the field of devices that prevent collapse of tubular structures of the body, such as endoluminal vascular prostheses or stent graft.

### INTRODUCTION

The present invention relates to an endoprosthesis for endovascular treatment of thoracic-abdominal aortic aneurysms or dissections and of abdominal aortic aneurysms or dissections which compromise the iliac arteries.

### STATE OF THE ART

The endovascular treatment of aortic aneurysm or dissection is performed by implanting a coated vascular endoprosthesis (minimally invasive device comprising a polymer-graft tube attached to a support-stent structure) that is intended to exclude or isolate the aneurysmal sac or dissection region and restore normal blood flow in the artery.

However, the incidence of aneurysms in regions where there are vital branches in the aorta (subclavian arteries, carotid arteries and visceral arteries) complicates the implantation of conventional stent graft, available in the medical devices market.

The vital branches cannot be obstructed, as this would result in branches-irrigated regions or organs failure.

Malina et al. (MAUNA M. et al., “EVAR and complex anatomy: An update on fenestrated and branched stent grafts”, 2008, Scandinavian Journal of Surgery, n.97: 195-204, 2008) estimate that 20% of patients who develop aortic aneurysms exhibit abdominal aortic aneurysm neck morphology which is inadequate for using standard or conventional endoprosthesis, that requires to cross the visceral branches to achieve an efficient sealing of the endoprosthesis, i.e., the isolation of the aneurysm.

Tsilimparis et al. (TSILIMPARIS, N.; RICOTTA II, J. J., “Type IV Thoracoabdominal Aneurysms: What's Next?”, 2012, Endovascular Today, March.) corroborate to the estimates of Malina et al., emphasizing that 20% of the patients with aortic aneurysms cannot be treated with commercially available devices due to the fact that the aneurysm compromises the visceral branches.

Accordingly, Murphy et al. (MURPHY, E. H. et al., “Fenestrated Endografting for the Treatment of Descending Thoracic Aneurysms”, 2009, Endovascular Today, January, pp. 26-33.) claim that more than 20% of the patients with abdominal aortic aneurysm could avoid open surgery and benefit from endovascular treatment if there were devices that could be used on the vital branches.

Efforts have been made to overcome these limitations by embedding fenestrations (windows or openings) or branches in the endoprosthesis during the manufacturing process—see Anderson (Anderson, J. L., “Fenestrated and branch aortic stent grafts”, Endovascular Today, April 2004, pp. 40-46) and Stanley et al. (Stanley, B. M.; Semmens, J. B.; Lawrence-Brown, M. M. D.; Goodman, M. A. and Hartley, D. E., “Fenestration in endovascular grafts for aortic aneurysm repair: new horizons for preserving blood flow in branch vessels”, J. Endovasc. Ther., 2001, v. 8, pp. 16-24).

Despite the satisfactory results, these devices require a high degree of customization, taking into consideration the anatomy of each patient, which significantly increases the cost of production of these devices and the waiting time to perform the procedure, since the device must be manufactured on demand.

A technical solution found for the maintenance of vital branches of the aorta is described by Kasirajan (Kasirajan, K., “Tandem Endografts for Type II TAAAs”, 2011, vol.10, n.5, pp. 30-34.) by treating the thoracoabdominal aortic aneurysm using numerous commercial endoprosthesis (off-the-shelf). Kasirajan (2011) uses a technique using arterial endoprosthesis simply connected (one inside the other) or connected in parallel, i.e., by placing one or two parallel endoprosthesis inside another straight or bifurcated arterial endoprosthesis.

According to Lobato et al. (Lobato, A. C. et al., “The sandwich technique: how to make it work for thoracoabdominal aneurysm exclusion”, Journal of Vascular and Endovascular Surgery, 2011, vol.18, pp-1-2.) and to Kolvenbach et al. (Kolvenbach, R. R. et al., “Urgent Endovascular Treatment of Thoraco-abdominal Aneurysm Using a Sandwich Technique and Chimney Grafts—A Technical Description”, European Journal of Vascular Surgery, 2010, xx, pp. 1-7.), the use of endoprosthesis in parallel (chimney, snorkel and sandwich) has been disseminated as a possible solution for the cases of extreme urgency because it makes use of conventional commercial endoprosthesis (off-the-shelf). However, as Tsilimparis and Ricotta II (2012) highlight, there are no medium- or long-term data confirming the effectiveness of these types of techniques, making its use in elective procedures still questionable in the medical area. Malina et al. (2008) warn that the chimney technique in parallel, when used in long lengths, can generate great channels between endoprostheses causing Type I endoleak.

Efforts in addressing the treatment both of aortic arch aneurysms and thoracoabdominal aneurysms were made by Chuter (WO2005027784), which aims to build already branched endoprostheses without the need for customization. As described by Chuter (2005) the lined stents have small predefined branches that are extended with other stents for the maintenance of aortic vital branches. However, as Chuter (2005) describes, different configurations are needed for different regions of the aorta, in other words, the solution is not universal. Chuter (2005) also describes some configurations of solutions for endoluminal prosthesis with multiple cylindrical threads, positioned in parallel within a larger cylinder, however there are spaces between the cylinders which may cause leaks between them and may affect the treatment. In another document, Chuter et al. (US20100312326) describe some configurations of solutions for modular bifurcated stents that are based on a cylindrical main body with internal cylinders connected and directed outwards the main cylinder turning into branches, similar to the configurations described by Greenberg (US20090048663). This solution can also present leaks in the connections of peripheral endoprostheses, or in the connection of the peripheral endoprostheses with collateral vessels, causing problems in aneurysm revascularization,

Parodi (WO2013071222) describes, in a generic manner, fenestrated and branched endoprostheses called “universal” for the treatment of aortic aneurysm. According to Parodi (WO2013071222), the solution is based on different configurations of cylinders of different diameters placed side by side in parallel along the aorta axis, sharing internal and external walls, depending on the configuration (Parodi, WO2013071222, [0095] p. 20). The solution of Parodi (2013) resembles the configurations described by Greenberg et al. (US20060247761). In the settings described by both, there may be problems of leak between cylinders connected laterally, as well as turbulence problems may occur with the blood flow due to perpendicular or inclined cylinders position with respect to the movement of blood in the artery. This can harm the development of visceral branches revascularization.

Hartley et al. (US20110257731) describe a coated stent graft for the treatment of aortic thoracic arch. The device consists of two or three tube portions, sutured to each other within a stent graft and sutured internally to the same stent graft, similar to the description of Parodi (2013). In another document, Hartley et al. (WO03082153) describe some configurations of arterial endoprosthesis coated with internal branches connected to the fenestrations, with the purpose of maintenance of vital branches of the aorta. Both documents exhibit the same above-mentioned problems, that is, there is a risk of leaks between cylinder connections, risks of turbulence in the blood stream due to the position of the internal cylinders and risk of leaks when connecting peripheral endoprosthesis with collateral vessels, causing aneurysm revascularization problems, making the treatment ineffective.

The generic configurations of the mentioned documents present similar features based on fenestrations in the main body and branches connected to the main body used for the coupling of other coated endoprosthesis and that, generally, in addition to the aforementioned problems, can even cause difficulties in the surgical procedure and in the patient's recovery. Even though being referred to as “universal”, the described configurations require positioning accuracy in connections with the visceral branches to reduce the likelihood of leaks.

Considering that there are numerous small aortic branches that keep the blood flow to the spinal cord, the placement of a thoracoabdominal endoprosthesis can cause postoperative paraplegia. None of the described configurations present an alternative for reducing the risk of patient's postoperative paraplegia.

As can be inferred from the above description, there is room for an endoluminal vascular prosthesis or coated arterial endoprosthesis that overcomes the disadvantages of the state of art and provides a precise and secure coupling device over the coated arterial endoprosthesis commercially available, providing greater efficiency of sealing of coupled joints, thus minimizing any possibility of leakage between connections while maintaining an internal blood flow without turbulence, assuring the patency rates of vital branches of the aorta, providing the surgeon with greater safety, agility and ensuring the effectiveness of the treatment to the patient.

### Objectives of the Invention

One of the objects of the present invention is to provide an endoprosthesis for endovascular treatment according to the features of claim 1. Another objective of this invention is to provide an endoprosthesis for endovascular treatment according to the features of claim X. Further features and detailing thereof are represented by dependent claims Y to Z.

### DESCRIPTION OF THE DRAWINGS

For a better understanding and visualization of the object of the present invention, it will now be described with reference to the attached drawings, representing the technical effect obtained by means of an exemplary embodiment, not limiting the scope of the present invention, wherein, schematically:

FIG. 1A: presents an anterosuperior view of a three-dimensional model of a preferred first embodiment of the object device of the present invention;

FIG. 1B: presents a detailing of the device in FIG. 1A;

FIG. 2A: presents a perspective top view of a three-dimensional model of the object device of the present invention;

FIG. 2B: presents a detailing of the device in FIG. 2A;

FIG. 3A: presents a perspective bottom view of a three-dimensional model of the object device of the present invention;

FIG. 3B: presents a detailing of the device in FIG. 3A;

FIG. 4A: presents a perspective view of a three-dimensional model of a further preferred embodiment of the object device of the present invention;

FIG. 4B: presents a detailing of the device in FIG. 4A;

FIG. 5A: presents a perspective top view of a three-dimensional model of the device in FIG. 4A;

FIG. 5B: presents a detailing of the device in FIG. 5A;

FIG. 6A: presents a perspective bottom view of a three-dimensional model of the device in FIG. 4A; and

FIG. 6B: presents a detailing of the device in FIG. 6A.

### FIGURES NUMERIC REFERENCES

1 endoprosthesis

2 proximal region

3 intermediate region

4 distal region

5 internal cylinder

6 opening

7 internal cylinder

8 opening

9 internal cylinder

10 opening

11 internal cylinder

12 opening

13 internal cylinder (fifth cylinder)

14 opening (of the fifth cylinder)

15 proximal flow input region

16 distal flow output region

17 proximal stent

18 distal stent

19 proximal radiopaque markers

20 distal radiopaque markers

50 endoprosthesis

51 proximal region

52 intermediate region

53 distal region

54 internal cylinder

55 opening

56 proximal flow input region

57 proximal stent

58 flow output

59 radiopaque marker

60 distal stent

### DETAILED DESCRIPTION OF THE INVENTION

### “Thoracoabdominal” Embodiment

The endoprosthesis (1) object of the present invention can be manufactured from a tubular coating—a biocompatible polymeric material (ePTFE, polyester or similar) graft with or without the support structure—biocompatible material (stainless steel, polymer, nitinol, molybdenum chromium steel or similar material) stent.

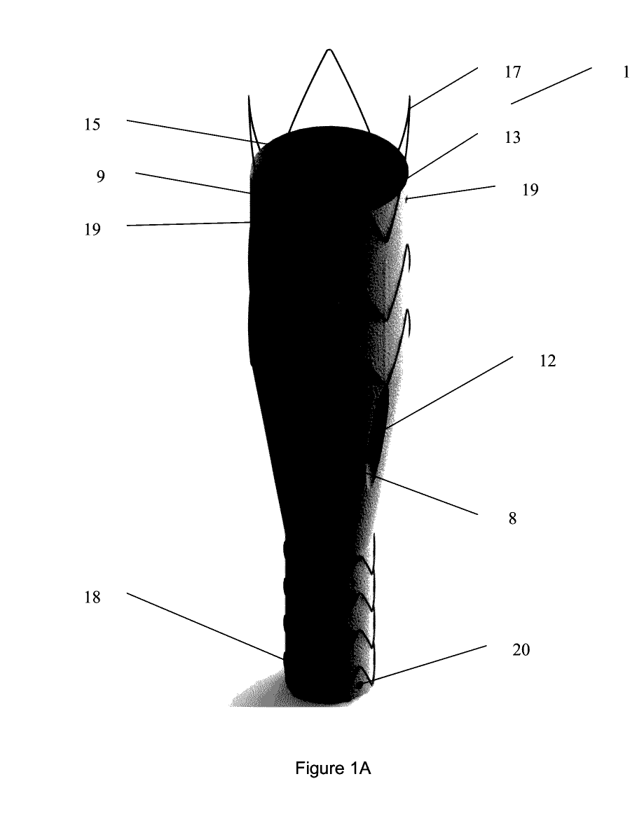

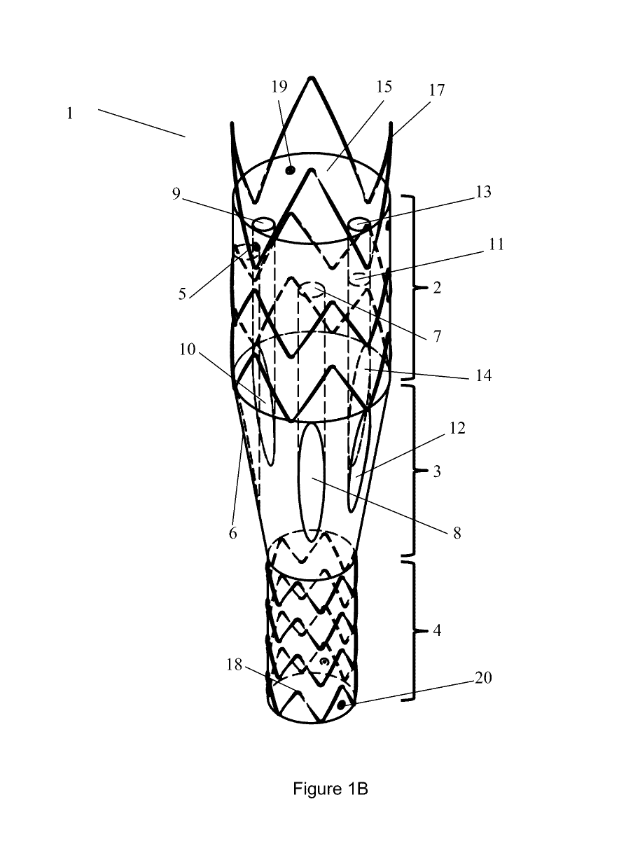

In a preferred embodiment of the invention, represented by FIGS. 1A to 3B, the endoprosthesis (1) according to the invention comprises three distinct sections being: a proximal region (2), an intermediate region (3) and a distal region (4). The endoprosthesis (1) is designed for the endovascular treatment of thoracic-abdominal aortic aneurysms or dissections.

The endoprosthesis (1) has a support structure—a proximal stent (17) and a distal stent (18)—here represented by several spaced apart rings in the form of the traditional “Gianturco z-stent” (see also U.S. Pat. No. 4,580,568). However, this structure can have a different or reduced configuration depending on the dimensions of the diameter, length and positioning location of the specific endoprosthesis.

For those skilled in the art, the multiple forms and materials configurations possible in the structures with stent (17, 18) are notorious. The traditional and conventional “z-stent” is characterized by having symmetrical apexes with generally equal curvature radius. As examples for illustration, the various stents used as support structure for the coated endoprosthesis described in WO2012/051532A2, WO2012/015670A1 and US2012/0165917A1, among others, can be mentioned.

Coated endoprosthesis diameters (1) are defined by the tubular coating—graft and can range from 18 to 60 mm. The length of the coated endoprosthesis (1), object of the present invention, can range from 60 to 250 mm.

The endoprosthesis (1) presents a proximal flow input region (15) and a distal flow output region (16). The endoprosthesis configuration (1) exhibits five internal cylinders (5), (7), (9), (11), and (13), internal to the main body and distributed along the inner wall thereof. These cylinders (5), (7), (9), (11), and (13) connect the flow of the proximal flow input (15) and release the flow through the respective openings (6), (8), (10), (12), and (14) in the intermediate region (3) of the endoprosthesis (1).

Due to the positioning of cylinders (5), (7), (9), (11), and (13), which is parallel to the direction of blood flow, turbulence problems are minimized or eliminated, providing security during the treatment and patient safety. The internal arrangement of cylinders (5), (7), (9), (11), and (13), independent and positioned in the direction of the blood flow, facilitates the visceral branches catheterization reducing the risks and the time of the surgical procedure. The length and arrangement of internal cylinders (5), (7), (9), (11), and (13) allows a large area of sealing and proximal fastening of peripheral endoprosthesis. These features contribute to the evolution of the visceral branches revascularization, making the procedure fast, safe and effective both for the surgeon and the patient.

The inputs of the internal cylinders (5), (7), (9), (11), and (13), as well as its openings (6), (8), (10), (12), and (14) in the intermediate region (3) of the endoprosthesis main body (1), may exhibit stent-type support structures (17) or metal or plastic rings to keep them open.

The openings (6), (8), (10), (12), and (14) of the internal cylinders (5), (7), (9), (11), and (13), arranged in the intermediate region (3), combined with the conicity of said intermediate region (3) of the main body, allow a greater space for the surgeon to manipulate between the endoprosthesis (1) and the wall of aorta. This space facilitates the catheterization of all visceral branches providing greater security and speed to the surgeon.

The openings (6), (8), (10), (12), and (14) are elliptical to facilitate the movement of the catheters to enable selectiveness in the branches and the peripheral stents adaptation to the different positions of the visceral branches. The large elliptical openings (6), (8), (10), (12), and (14) also provide a good zone for manipulation, there having no need for accuracy of positioning and angulation to the height of the output for said visceral branches.

These features contribute to a greater safety and efficiency in treating for both the surgeon and the patient.

The joining of the internal cylinders (5), (7), (9), (11), and (13) to the inner wall of the endoprosthesis main body (1), as well as the joining of its openings (6), (8), (10), (12), and (14) to the endoprosthesis main body (1), can be accomplished by means of suture, bonding or welding.

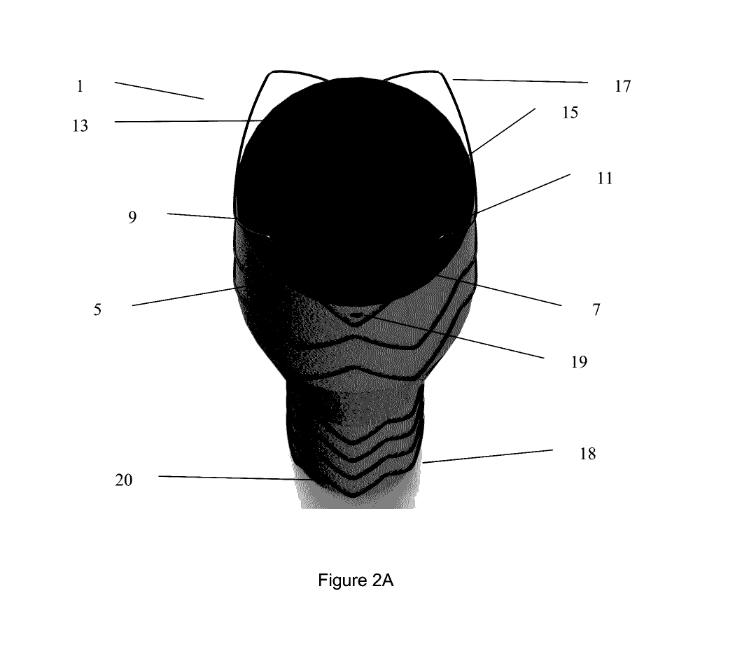

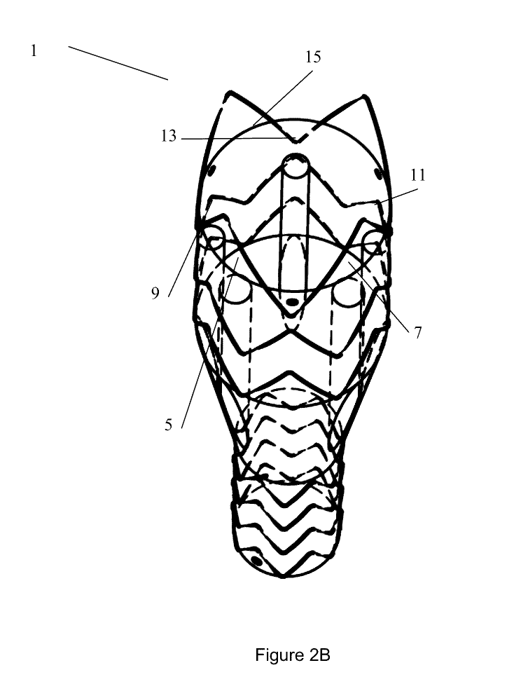

FIG. 2B shows the detail of the endoprosthesis (1) in the proximal region (2), highlighting the proximal flow input of the input region (15) and the inputs of the five internal cylinders (5), (7), (9), (11), and (13).

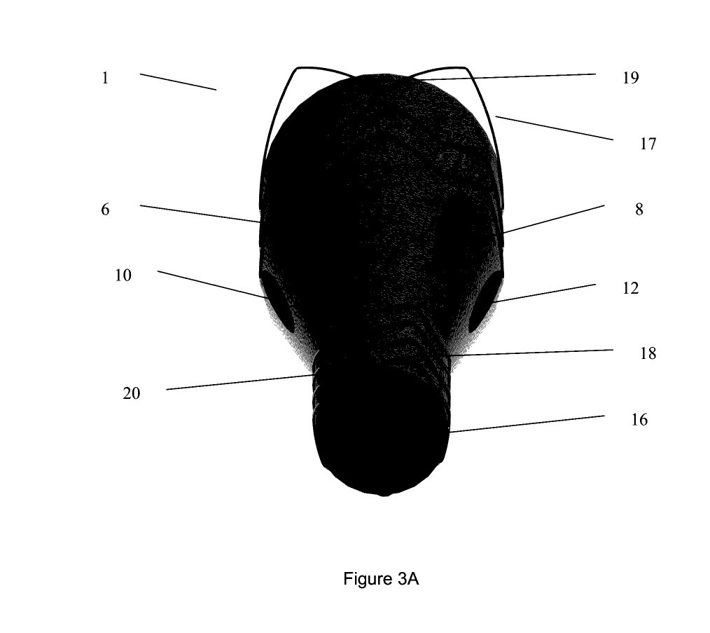

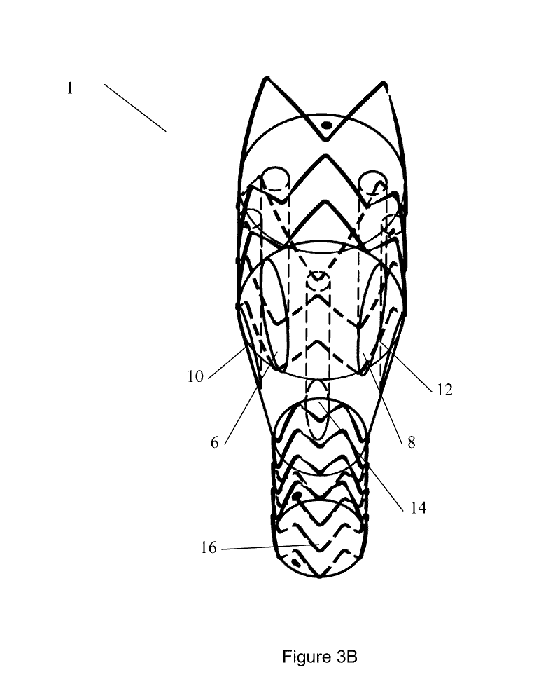

FIG. 3B shows in detail the intermediate region (3) and the distal region (4) of the endoprosthesis (1) showing the openings (6), (8), (10), (12), and (14) of the internal cylinders (5), (7), (9), (11), and (13), as well as the distal flow output region (16) in the distal region (4).

The presence of the fifth internal cylinder (13) with flow and opening input (14) represents a relevant differential of the endoprosthesis (1) of the present invention over the state of art, providing a novel technical effect allowing the maintenance of the blood flow also to the spinal cord, in order to reduce the risk of postoperative paraplegia. This fifth internal cylinder (13) can be occluded a few days after the surgical procedure, in case it is not usefulness anymore and in case the risk of paraplegia has decreased or has been discarded. These features contribute to greater safety and efficiency in treating for both the surgeon and the patient, eliminating potential significant sequelae of the treatment.

Finally, it should be noted that the endoprosthesis (1) should preferably exhibit radiopaque markers (19, 20) to facilitate its viewing by fluoroscopy during the implantation procedure. The proximal (19) and distal radiopaque markers (20) are represented in the configuration of the endoprosthesis (1), however, as it is obvious to any person skilled in the art, the radiopaque markers (19, 20) can assume different geometries and can be manufactured in different materials, as well as they can assume different positions in the endoprosthesis (1), for example, in the internal cylinders inputs (5), (7), (9), (11), and (13) and/or the respective openings (6), (8), (10), (12), and (14) and/or in the proximal flow input region (15) and/or distal flow output region (16) of the endoprosthesis main body (1).

### “Iliac” Embodiment

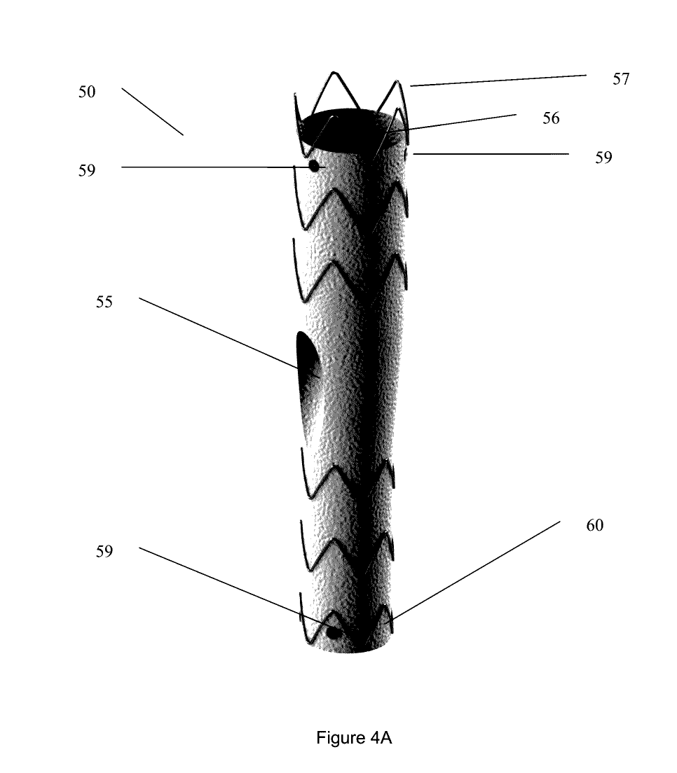

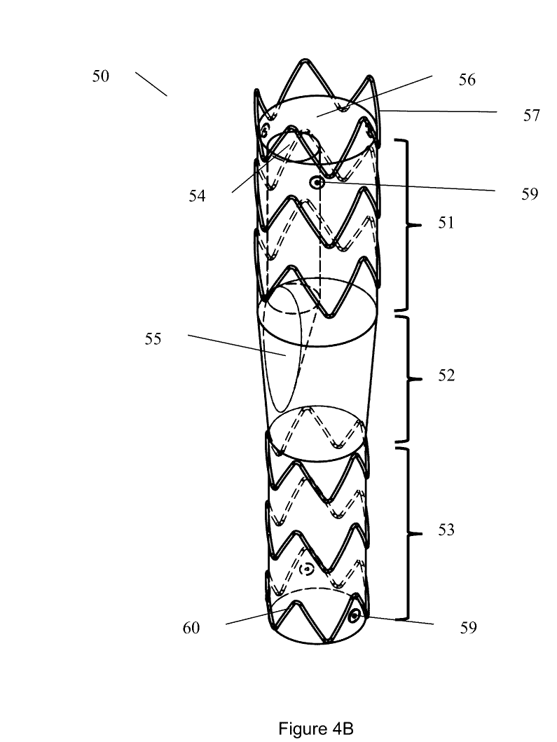

In a further preferred embodiment of the invention, represented by FIGS. 4A to 6B, the endoprosthesis (50) according to the invention comprises three distinct sections being: a proximal region (51), an intermediate region (52) and a distal region (53). The endoprosthesis (50) is designed to be used in abdominal aortic aneurysms that compromise the region of the iliac arteries.

The endoprosthesis (50) has a support structure—proximal stent (57) and a distal stent (60)—comprising several spaced apart rings in the form of the traditional “Gianturco z-stent” (see also U.S. Pat. No. 4,580,568). However, this structure can have a different or reduced configuration depending on the dimensions of the diameter, length and positioning location of the specific endoprosthesis.

For those skilled in the art, the multiple forms and materials configurations possible in the structures with stent (57, 60) are notorious. The traditional and conventional “z-stent” is characterized by having symmetrical apexes with generally equal curvature radius. As examples for illustration, the various stents used as support structure for the coated endoprosthesis described in WO2012/051532A2, WO2012/015670A1 and US2012/0165917A1, among others, can be mentioned.

The coated endoprosthesis diameters (50) are defined by the tubular coating—graft and can range from 5 to 25 mm. The length of the coated endoprosthesis (50), object of the present invention, can range from 40 to 200 mm.

The endoprosthesis (50) further presents a proximal flow input region (56). In the endoprosthesis (50) configurations there is an inner cylinder (54), internal to the main body, positioned along the inner wall thereof. Said cylinder (54) connects the proximal flow input region flow (56) and releases the flow through the opening (55), placed in the intermediate region (52) of the endoprosthesis (50).

The internal cylinder input (54) can present proximal stent-type support structures (57) or metal or plastic rings to keep it open, as well as the opening (55) thereof in the intermediate region (52) of the endoprosthesis main body (50).

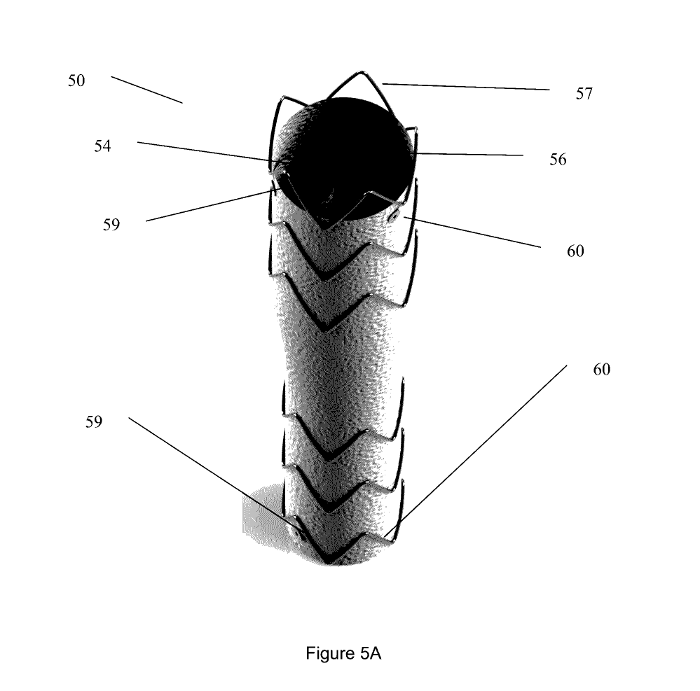

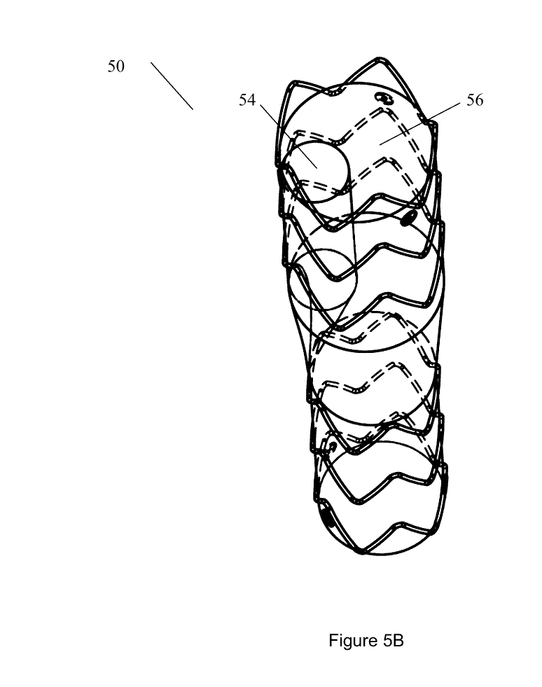

FIG. 5B presents the detail of the endoprosthesis (50) in the proximal region (51), showing the proximal flow input region (56) in the main body and the internal cylinder (54).

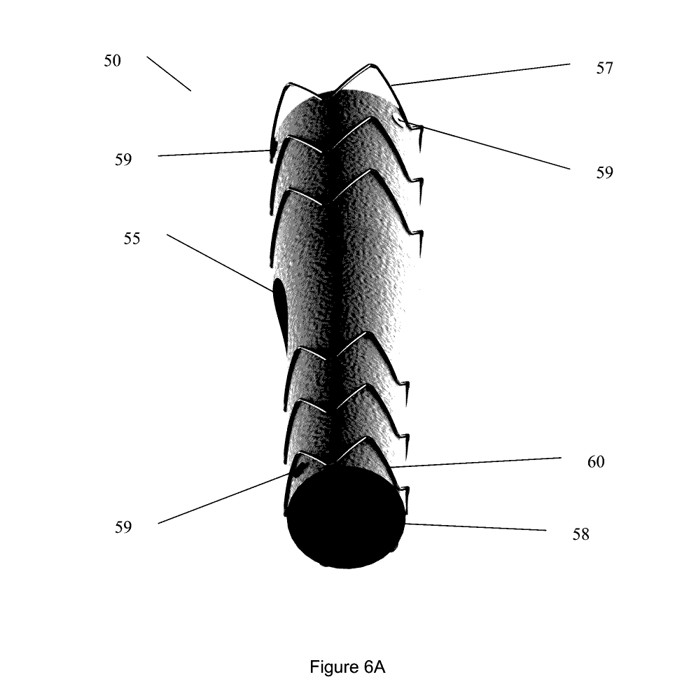

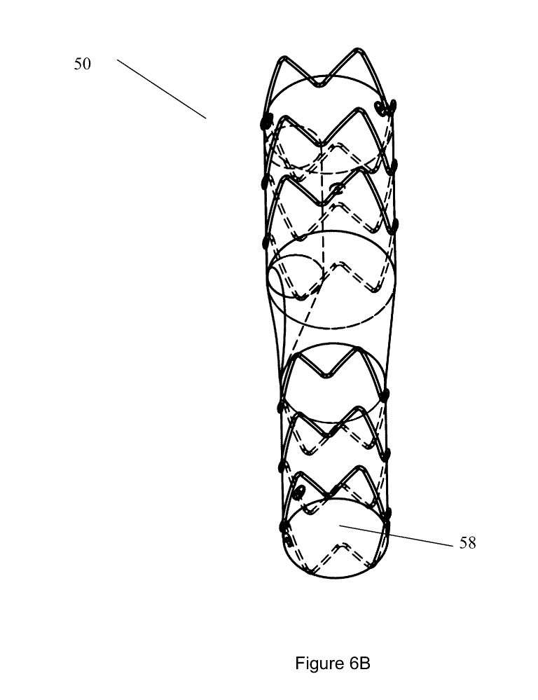

FIG. 6B shows in detail the distal region (53) of the endoprosthesis (50) presenting flow output (58) in the distal region (53).

As the endoprosthesis (50) is designed for use in the iliac artery, it has only one internal cylinder (54) that will be used in the internal iliac artery catheterization. The endoprosthesis (50) feature of having the inner cylinder (54) parallel to the blood flow prevents turbulence in the bloodstream. The internal cylinder (54) in longitudinal position also acts as facilitator for the peripheral vessels catheterization, and its large internal area allows an effective sealing of peripheral endoprosthesis that is/will be connected to the endoprosthesis (50). The elliptical, big opening (55), and the conical shape of the main body provide the surgeon with safety to position and manipulate when catheterizing the peripheral vessel.

Said features contribute to improved safety and efficiency in treating for both the surgeon and the patient, eliminating potential risks of treatment, facilitating the manufacture and provision of a universal endoprosthesis.

Finally, the endoprosthesis (50) should preferably exhibit radiopaque markers (59) to facilitate its viewing by fluoroscopy during the implantation procedure. The radiopaque marker (59) is represented in the endoprosthesis (50) settings, but as it is obvious to any person skilled in the art, radiopaque markers (59) can assume different geometries and be manufactured in different materials, as well as some may assume different positions in the endoprosthesis (50), for example, in the internal cylinder input (54) and/or opening (55) thereof and/or in the proximal flow input region (56) of the endoprosthesis main body (50).

### Final Considerations

As can be inferred from the above description, the endoprosthesis (1, 50) according to the invention has a new and inventive technical effect, resulting in a better performance of its main functions as well as greater security for the surgeon, and consequently greater effectiveness in the treatment of patients' aortic disease.

The solution described in this document therefore presents some significant advantages over the state of art devices described, namely:
a) A conical main body, which provides the surgeon with a larger manipulation space between the endoprosthesis (1, 50) and the wall of the aorta. Said space facilitates the catheterization of all visceral branches providing greater security and speed to the surgeon;
b) The extent of the internal cylinders (5), (7), (9), (11), and (13), independent and positioned in the direction of the blood flow, allows an efficient sealing and proximal fastening of peripheral endoprosthesis, promoting both the procedure and the evolution of visceral branches revascularization;
c) The possibility of being an off-the-shelf endoprosthesis (1, 50), with standard sizes for universal use and for use in emergencies;
d) The internal cylinders (5), (7), (9), (11), and (13) enable a pre-catheterization with small-diameter stripe guides, facilitating the visceral branches catheterization and decreasing the risks and the time of the surgical procedure;
e) The large elliptical openings (6), (8), (10), (12), and (14) facilitate the movement of the catheters to enable selectiveness in the branches and the peripheral endoprosthesis adaptation to the different positions of the visceral branches;
f) There is no need for customizations of the endoprosthesis (1, 50) as it is the case of commercially available fenestrated endoprosthesis;
g) Due to the positioning of internal cylinders (5), (7), (9), (11), and (13), and of the large elliptical openings (6), (8), (10), (12), and (14), there is no need for positioning accuracy and angulation at the height of the output for the visceral branches;
h) The endoprosthesis (1) allows to treat thoracoabdominal aneurysms with four or less visceral branches with the same endoprosthesis (1);
i) The presence of a fifth cylinder (13) allows to keep a blood flow to spinal cord, in order to decrease the risk of paraplegia, wherein the opening (14) of which can be occluded after a few days if it is not useful anymore and if the risk of paraplegia has decreased;
j) Adjustment of the main types of commercially available endoprosthesis to complete the procedure is allowed.

The solution described in this document also has the following advantages in surgical practice related to parallel techniques that have been increasingly used to treat aneurysms that compromise branched aortic regions:
a) Peripheral endoprosthesis implantation and catheterization in connections to visceral branches, one at a time, without need to catheterize all at the same time, as done in parallel techniques like, chimney, snorkel, sandwich and octopus, are enabled;
b) Only one access to upper members and use of lower-profile sheath or introducer, decreasing local risks, like arterial dissections, thrombosis, artery rupture, perforations, embolisms, difficulty of passing devices and decreased risk of systemic complications as stroke, are required;
c) Decreased risk of proximal leakage when compared to parallel techniques because of the effective and appropriate sealing of the peripheral endoprostheses in the internal cylinders (5), (7), (9), (11), and (13) since it presents a large area of sealing.

### CONCLUSION

It will be easily understood by those skilled in the art that changes can be made in the present invention without departing from the concepts exposed in the above description. These modifications must be regarded as comprised by the scope of the present invention. Consequently, the particular embodiments previously described in detail are only illustrative and exemplary and are not restrictive as to the scope of the present invention, to which the full extent of the appended set of claims and any and all correspondents thereof should be given.

1. Endoprosthesis for endovascular treatment of thoracic-abdominal aortic aneurysms or dissections, said endoprosthesis (1) comprising a proximal region (2), an intermediate region (3) and a distal region (4), wherein it has five internal cylinders (5), (7), (9), (11), and (13), parallel to the longitudinal axis of the endoprosthesis (1), said internal cylinders (5), (7), (9), (11), and (13) being provided with elliptical openings (6), (8), (10), (12), and (14) arranged in said intermediate region (³).

2. Endoprosthesis for endovascular treatment of thoracic-abdominal aortic aneurysms or dissections, said endoprosthesis (50) comprising a proximal region (51), an intermediate region (52) and a distal region (53), wherein it has an internal cylinder (54), parallel to the longitudinal axis of the endoprosthesis (50), said internal cylinder (54) being provided with and elliptical opening (55) in said intermediate region (52).
